(19) United States
(12) Patent Application Publication
(10) Pub. No.: US 2018/0036447 A1
(43) Pub. Date: Feb. 8, 2018

(54) Fragrance Releasing Article with A Decorative Element

(71) Applicant: Beauty Touch Co., Ltd, Taipei (TW)

(72) Inventor: Chien-Yi Lin, Taipei (TW)

(21) Appl. No.: 15/415,139

(22) Filed: Jan. 25, 2017

(30) Foreign Application Priority Data
Aug. 8, 2016 (TW) 105125195

(51) Int. Cl. A61L 9/12; A47G 1/06

(52) U.S. Cl. CPC A61L 9/12; A47G 1/0616; A61L 2209/15

(57) ABSTRACT

A fragrance releasing article, comprising a rear panel and a front panel, the rear panel having an opening, a decorative element receiving part and a fragrance releasing element receiving part provided in the opening for receiving a fragrance releasing element and a decorative element, respectively; the front panel removably covering and attached to the opening of the rear panel, the front panel comprising a decorative element covering part and a fragrance releasing element covering part for covering the fragrance releasing element receiving part and the decorative element receiving part, respectively, the fragrance releasing element covering part having at least one opening for releasing the fragrance of the fragrance releasing element, and the decorative element covering part having a display window for displaying the decorative element.

### FIELD OF THE INVENTION

The present invention relates to a fragrance releasing article, and more particular to a fragrance releasing article with a decorative element.

### BACKGROUND OF THE INVENTION

Fragrance makes people feel pleasant. Recently, people like to use a variety of fragrance devices to improve the indoor odor and increase the enjoyment of life. Common fragrance devices are essential oil lamps, scented candles and a variety of volatile fragrance gel or scented disc. However, fragrance devices such as the essential oil lamps or scented candles need to be ignited or plugged in to heat to evaporate their aromatic ingredients into the air. The use of these types of fragrance devices is either electrical energy-consuming, or the user should always pay attention to the surroundings and the burning conditions to avoid accidents. In contrast, the use of volatile fragrance gel or scented disc is not electrical energy-consuming, and the user does not need to pay attention to the safety issues.

Fragrance gel or scented disc is usually placed in a fragrance device. The outer package of some of the commercially available fragrance gel or scented disc has a simple design which can be directly open and used. However, the fragrance device and the outer package hardly have a decorative appearance or the appearance does not match with the interior decoration, which makes them look unfit to the interior decoration. Besides, the appearance can not be changed optionally to fit a newly renovated interior decoration.

### SUMMARY OF THE INVENTION

Accordingly, in order to solve the foregoing problem that the fragrance device hardly match with the interior decoration, the present invention provides an fragrance releasing article with a decorative element, in which the fragrance releasing element and the decorative element can be replaced optionally, so that the fragrance releasing article can be taken as an ornament while providing fragrance to the indoor air, and therefore the fragrance releasing article has the advantage of being decorative, practical, environmentally friendly, and flexible in arrangement.

To reach the objects stated above, the present invention provides a fragrance releasing article which comprises a rear panel and a front panel. The rear panel has an opening, and a decorative element receiving part and a fragrance releasing element receiving part are provided in the opening for receiving a fragrance releasing element and a decorative element, respectively. The front panel is removably covering and attached to the opening of the rear panel. The front panel comprises a decorative element covering part and a fragrance releasing element covering part for covering the fragrance releasing element receiving part and the decorative element receiving part, respectively. The fragrance releasing element covering part has at least one opening for releasing the fragrance of the fragrance releasing element, and the decorative element covering part has a display window for displaying the decorative element.

In implementation, the fragrance releasing article further comprises a carrier provided in the decorative element receiving part for carrying the decorative element.

In implementation, the carrier has a positioning part for positioning the decorative element.

In implementation, the decorative element is a photo or a picture.

In implementation, the fragrance releasing article further comprises a transparent element covering the display window of the decorative element covering part.

In implementation, the transparent element is a glass plate or an acrylic plate.

In implementation, a hanging hole is provided on the rear panel for hanging the fragrance releasing article.

In implementation, the fragrance releasing article further comprises a support element attached to the rear panel with one end of the support element for supporting the fragrance releasing article.

In implementation, the support element is pivotally connected to the rear panel with one end of the support element, so that the other end of the support element is movable relative to the rear panel.

In implementation, the fragrance releasing element is volatile in room temperature to release the fragrance of the fragrance releasing element.

The present invention will be understood more fully by reference to the detailed description of the drawings and the preferred embodiments below.

### BRIEF DESCRIPTION OF DRAWINGS

FIG. 1 is a exploded view of an embodiment of a fragrance releasing article provided by the present invention.

FIG. 2A is a front view of an embodiment of a fragrance releasing article provided by the present invention.

FIG. 2B is a rear view of an embodiment of a fragrance releasing article provided by the present invention.

FIG. 3 is a exploded view of another embodiment of a fragrance releasing article provided by the present invention.

FIG. 4A is a front view of another embodiment of a fragrance releasing article provided by the present invention.

FIG. 4B is a side view of another embodiment of a fragrance releasing article provided by the present invention.

FIG. 5A is a schematic drawing of an embodiment of a fragrance releasing article provided by the present invention.

FIG. 5B is a schematic drawing of another embodiment of a fragrance releasing article provided by the present invention.

### DETAILED DESCRIPTIONS OF PREFERRED EMBODIMENTS

The present invention provides a fragrance releasing article, which comprises a rear panel and a front panel. The rear panel has an opening, and a decorative element receiving part and a fragrance releasing element receiving part are provided in the opening for receiving a fragrance releasing element and a decorative element, respectively. The front panel is removably covering and attached to the opening of the rear panel. The front panel comprises a decorative element covering part and a fragrance releasing element covering part for covering the fragrance releasing element receiving part and the decorative element receiving part, respectively. The fragrance releasing element covering part has at least one opening for releasing the fragrance of the fragrance releasing element, and the decorative element covering part has a display window for displaying the decorative element.

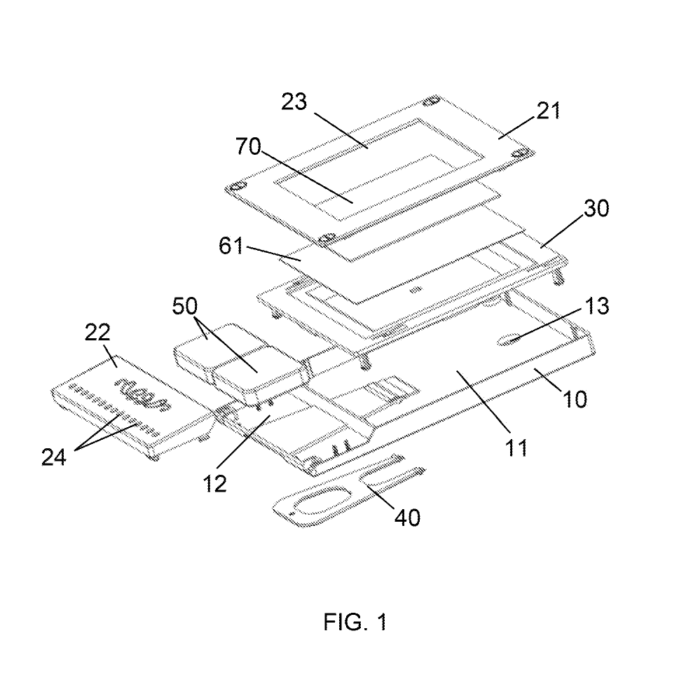

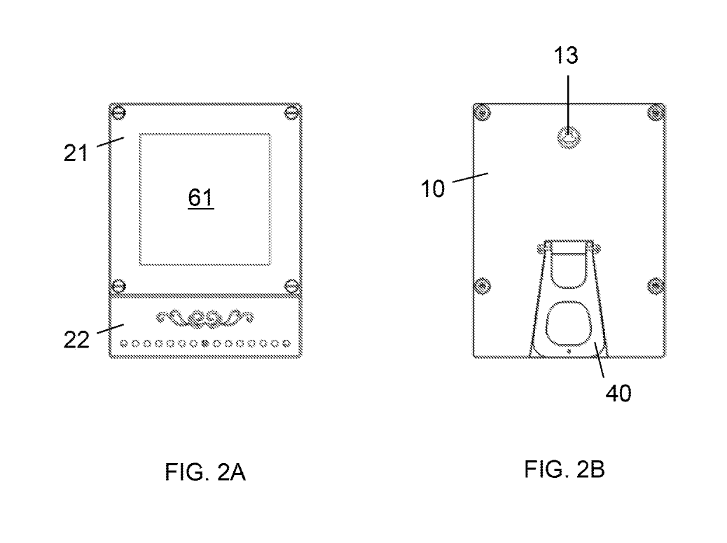

FIGS. 1, 2A, and 2B show an embodiment of a fragrance releasing article provided by the present invention. The fragrance releasing article comprises a rear panel 10, a carrier 30, a decorative element covering part 21 and a fragrance releasing element covering part 22. The rear panel 10 has an opening, and a decorative element receiving part 11 and a fragrance releasing element receiving part 12 are provided in the opening. The fragrance releasing element covering part 22 is removably covering and attached to the fragrance releasing element receiving part 12 and forming a fragrance releasing element receiving space for receiving at least one fragrance releasing element 50. The fragrance releasing element covering part 22 has at least one opening 24 for releasing the fragrance of the fragrance releasing element 50. The carrier 30 is provided in the decorative element receiving part 11 for carrying a decorative element 61. The decorative element covering part 21 has a display window 23 for displaying the decorative element 61.

In one embodiment, the decorative element 61 may be a photo or a picture. The decorative element covering part 21 may comprises a transparent element 70 covering the display window 23 of the decorative element covering part 21. In one embodiment, the transparent element 70 may seal the display window 23 to prevent the decorative element 61 from physical or chemical contamination. The transparent element 70 may be a glass plate, an acrylic plate, or a thin plate made of other transparent material.

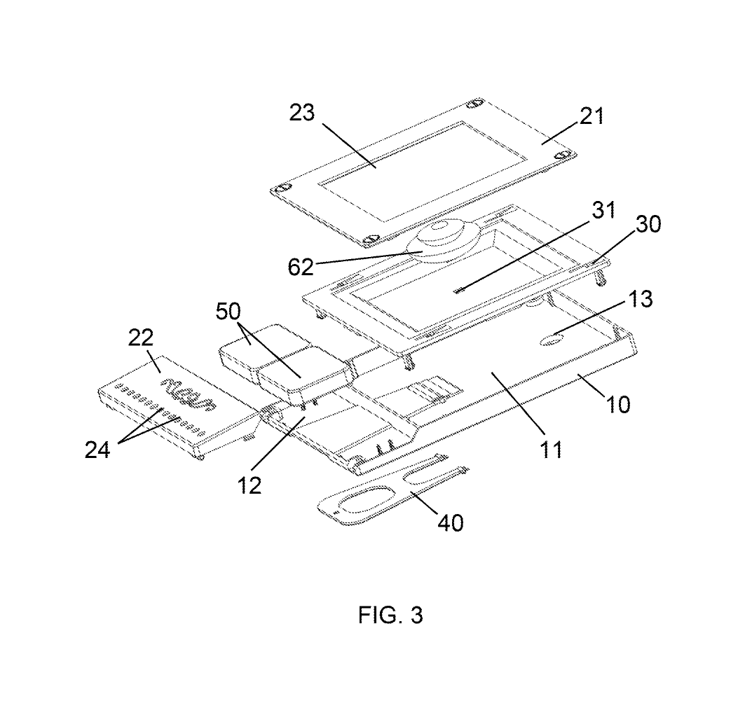

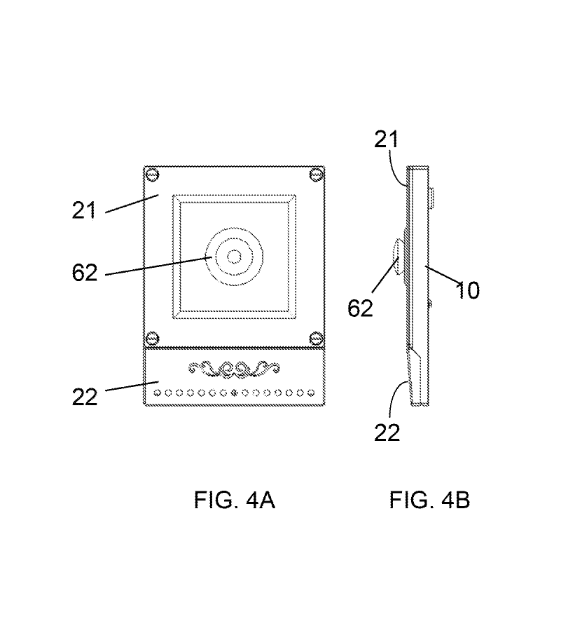

FIGS. 3, 4A, and 4B show another embodiment of a fragrance releasing article provided by the present invention. The fragrance releasing article comprises a rear panel 10, a carrier 30, a decorative element covering part 21 and a fragrance releasing element covering part 22. The rear panel 10 has an opening, and a decorative element receiving part 11 and a fragrance releasing element receiving part 12 are provided in the opening. The fragrance releasing element covering part 22 is removably covering and attached to the fragrance releasing element receiving part 12 and forming a fragrance releasing element receiving space for receiving at least one fragrance releasing element 50. The fragrance releasing element covering part 22 has at least one opening 24 for releasing the fragrance of the fragrance releasing element 50. The carrier 30 is provided in the decorative element receiving part 11. The carrier 30 has a positioning part 31 for positioning a decorative element 62. The decorative element covering part 21 has a display window 23 for displaying the decorative element 62.

In one embodiment, the decorative element 62 may be a stereo object. In one embodiment, the decorative element 62 may comprise an engaging element for engaging and positioning to the positioning part 31 of the carrier 30. In one embodiment, the positioning part 31 of the carrier 30 may be a positioning hole. The engaging element of the decorative element 62 may engage to the positioning hole to position the decorative element 62.

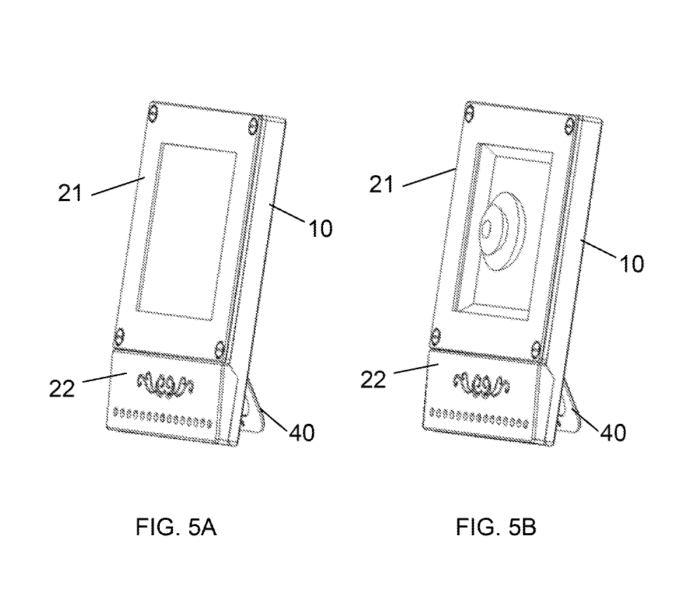

In one embodiment, a hanging hole 13 may be provided on the rear panel 10 of the fragrance releasing article for hanging the fragrance releasing article. In one embodiment, the fragrance releasing article may further comprise a support element 40 attached to the rear panel 10 with one end of the support element for supporting the fragrance releasing article. In one embodiment, the support element 40 is pivotally connected to the rear panel 10 with one end of the support element 40, so that the other end of the support element 40 is movable relative to the rear panel 10 to fold or unfold the support element 40. As shown in FIGS. 5A and 5B, the fragrance releasing article is supported as one end of the support element 40 is unfolded from the rear panel 10.

In one embodiment, the decorative element may be directly received in the decorative element receiving part of the rear panel. The rear panel may comprise a positioning part for positioning the decorative element. In the abovementioned embodiment, the decorative element covering part and the fragrance releasing element covering part are separate structural parts. In one embodiment, the decorative element covering part and the fragrance releasing element covering part may be constructed integrally by the front panel.

In the embodiments provided by the present invention, the front panel and the rear panel can be repeatedly engaged and disengaged with each other. In one embodiment, the rear panel may comprise one or more rear panel engaging members, and the front panel may comprise one or more corresponding front panel engaging members for engaging with the rear panel engaging members of the rear panel. In the embodiments provided by the present invention, the carrier and the decorative element covering part can be repeatedly engaged and disengaged with each other. In one embodiment, the carrier may comprise one or more carrier engaging members, and the decorative element covering part may comprise one or more corresponding decorative element covering part engaging members for engaging with the carrier engaging members of the carrier.

In the embodiments provided by the present invention, the fragrance releasing element is volatile in room temperature to release the fragrance of the fragrance releasing element. In one embodiment, the fragrance releasing element is an solid scented disc, an aromatic bag, or a bundle of aromatic herbs, that can spontaneously releases fragrance.

The present invention has the following advantages:

1. The fragrance releasing article provided by the present invention is decorative and can release fragrance. The fragrance releasing article can be taken as an ornament while providing fragrance to the indoor air, and therefore it is decorative and practical.

2. The structural members of the fragrance releasing article provided by the present invention can be repeatedly engaged and disengaged, so that the user can replace the fragrance releasing element and the decorative element freely. The fragrance releasing article is reusable, and therefore it is environmentally friendly and flexible in arrangement.

3. The fragrance releasing article provided by the present invention comprises separate fragrance releasing element receiving part and decorative element receiving part, so that contamination to the decorative element by the volatile ingredient releasing from the fragrance releasing element can be avoided.

4. The fragrance releasing article provided by the present invention can be arranged by hanging or placing on a platform, and therefore it is flexible in arrangement.

To sum up, the fragrance releasing article provided by the present invention can indeed meet its anticipated objective to be taken as an ornament while providing fragrance to the indoor air. The fragrance releasing article provided by the present invention is therefore decorative, practical, environmentally friendly, and flexible in arrangement.

The description referred to in the drawings and stated above is only for the preferred embodiments of the present invention. Many equivalent local variations and modifications can still be made by those skilled at the field related with the present invention and do not depart from the spirit of the present invention, so they should be regarded to fall into the scope defined by the appended claims.

What is claimed is:

1. A fragrance releasing article, comprising:
a rear panel, having an opening, a decorative element receiving part and a fragrance releasing element receiving part provided in the opening for receiving a fragrance releasing element and a decorative element, respectively; and
a front panel, removably covering and attached to the opening of the rear panel, the front panel comprising a decorative element covering part and a fragrance releasing element covering part for covering the fragrance releasing element receiving part and the decorative element receiving part, respectively, the fragrance releasing element covering part having at least one opening for releasing the fragrance of the fragrance releasing element, and the decorative element covering part having a display window for displaying the decorative element.

2. The fragrance releasing article according to claim 1, further comprising a carrier provided in the decorative element receiving part for carrying the decorative element.

3. The fragrance releasing article according to claim 2, wherein the carrier has a positioning part for positioning the decorative element.

4. The fragrance releasing article according to claim 2, wherein the decorative element is a photo or a picture.

5. The fragrance releasing article according to claim 4, further comprising a transparent element covering the display window of the decorative element covering part.

6. The fragrance releasing article according to claim 5, wherein the transparent element is a glass plate or an acrylic plate.

7. The fragrance releasing article according to claim 1, wherein a hanging hole is provided on the rear panel for hanging the fragrance releasing article.

8. The fragrance releasing article according to claim 1, further comprising a support element attached to the rear panel with one end of the support element for supporting the fragrance releasing article.

9. The fragrance releasing article according to claim 8, wherein the support element is pivotally connected to the rear panel with one end of the support element, so that the other end of the support element is movable relative to the rear panel.

10. The fragrance releasing article according to claim 1, wherein the fragrance releasing element is volatile in room temperature to release the fragrance of the fragrance releasing element.
